(19) United States
(12) Patent Application Publication
(10) Pub. No.: US 2024/0197807 A1
(43) Pub. Date: Jun. 20, 2024

(54) WATER SOLUBLE FORMULATIONS CONTAINING COENZYME-Q10 AND ASHWAGANDHA ROOT EXTRACT

(71) Applicant: NEXT REMEDIES INC., Toronto (CA)

(72) Inventors: Siyaram PANDEY, LaSalle (CA); Caleb VEGH, LaSalle (CA)

(21) Appl. No.: 18/553,328

(22) PCT Filed: Sep. 30, 2022
(86) PCT No.: PCT/CA2022/050477

§ 371 Date: Sep. 29, 2023

Related U.S. Application Data
(60) Provisional application No. 63/168,016, filed on Mar. 30, 2021.

(51) Int. Cl. A61K 36/81; A61K 31/122; A61K 47/22

(52) U.S. Cl. CPC A61K 36/81; A61K 31/122; A61K 47/22; A61K 2236/333

(57) ABSTRACT

The present application relates to water-soluble formulations. More specifically, the present application relates to formulations comprising at least one of Coenzyme-Q10 and an ashwagandha root extract and a solubilizing agent. More specifically, the present application relates to formulations comprising water-solubilized Coenzyme-Q10 in combination with alcohol-extracted ashwagandha root extract; water-solubilized alcohol-extracted ashwagandha root extract; or water-solubilized Coenzyme-Q10 in combination with water-solubilized alcohol-extracted ashwagandha root extract. The formulations of the present application are useful for treating reducing or preventing a neurological diseases or disorders.

### CROSS-REFERENCE TO RELATED APPLICATIONS

The present application claims priority to U.S. Provisional Patent Application No. 63/168,016, which was filed Mar. 30, 2021, the content of which is incorporated herein by reference in its entirety.

### FIELD

The present application is in the field of water-soluble formulations. More specifically, the present application relates to formulations comprising at least one of Coenzyme-Q10 and an ashwagandha root extract, and a solubilizing agent.

### BACKGROUND

Brain cells are vulnerable to oxidative stress, inflammation, mitochondrial dysfunction and accumulation of dysfunctional proteins. Indeed, these are the biochemical etiologies for common brain diseases, such as Alzheimer's and Parkinson's disease (AD and PD). Although good progress has been made in providing a symptomatic relief with dopamine supplements and deep brain stimulation, there is no known available remedy to stop the progression of these diseases. There are several biochemical mechanisms implicated in the progression of PD and AD including: oxidative stress, mitochondrial dysfunction, autophagy/proteosome deficiency, and neuroinflammation.

Ashwagandha (Withania somnifera) is a plant of the nightshade family that has been used in Ayurveda (traditional Indian school of medicine) as a nerve tonic for general debility, nervous exhaustion, insomnia, and memory impairment. Past studies showed that various root extracts of ashwagandha were able to target several pathologies of PD including oxidative stress and neuroinflammation. Unfortunately, the doses used for the extract were significantly high and unrealistic for therapeutic development.

Coenzyme-Q10, or ubiquinone-10, is naturally biosynthesized in most human tissue. Coenzyme-Q10 is currently sold as a dietary supplement. While previous studies with Coenzyme-Q10 showed therapeutic efficacy, the oral doses were much too high to be used as a therapeutic, mainly due to its poor water-solubility.

Coenzyme-Q10 and lipophilic compounds in Ashwagandha extracts have both shown some neuroprotective effects in rodent models of AD and PD, but effective doses were extremely high, for example in the range of 1000 mg/kg/day.

In view of the above, there is a need to develop formulations with good bioavailability for neuroprotection, that could improve brain health in patients with neurological disorders, such as AD and PD.

### SUMMARY

It has been surprisingly shown herein that formulations of the present application provide neuroprotective properties. The formulations of the present application including water-solubilized compounds further provide for increased bioavailability of the compounds. Comparable formulations did not display the same properties, highlighting the surprising results obtained with the water-solubilized formulations of the application.

Accordingly, the present application includes a formulation comprising:
(i) Coenzyme-Q10 water-solubilized with at least one solubilizing agent of Formula (I):

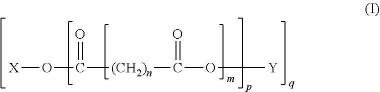

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
   (ii) an alcohol-extracted ashwagandha root extract.

The present application further includes a formulation comprising:
(i) an alcohol-extracted ashwagandha root extract water-solubilized with at least one solubilizing agent of Formula (I):

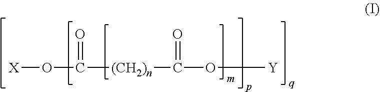

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2.

The present application also includes a formulation comprising:
(i) Coenzyme-Q10 water-solubilized with at least one solubilizing agent of Formula (I):

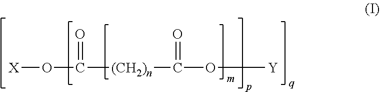

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
(ii) an alcohol-extracted ashwagandha root extract water-solubilized with at least one solubilizing agent of Formula (I).

Also provided is an emulsion comprising the formulation of the present application dispersed in water.

Further included is a method for treating, reducing or preventing a neurological disease or disorder in a subject in need thereof, comprising administering a pharmaceutically effective amount of a formulation of the present application to the subject.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF DRAWINGS

The embodiments of the application will now be described in greater detail with reference to the attached drawings in which:

FIG. 1 shows images of immunohistochemical staining of midbrain sections showing tyrosine hydroxylase (TH) positive neurons, at 25× (scale bar=250 microns) and 100× (scale bar=50 microns) for combined water-solubilized Coenzyme-10 and ashwagandha extract, according to exemplary embodiments of the application.

FIG. 2 shows A) images of immunofluorescent staining of the SN in midbrain sections probing for oxidative stress marker and lipid peroxidation by-product 4-hydroxynonenal (4-HNE) and apoptosis regulator CARP1 (scale bar=100 microns); B) quantification of 4-HNE corrected total fluorescence; and C) quantification of CARP1 corrected total fluorescence, for combined water-solubilized Coenzyme-10 and ashwagandha extract, according to exemplary embodiments of the application.

FIG. 3 shows A) images of immunofluorescent staining of the SN in midbrain sections probing for beclin-1, a major regulator of autophagy and Iba-1 (ionized calcium-binding adapter molecule 1), a marker of microglia activation (scale bar=100 microns); and B) quantification of corrected total fluorescence for beclin-1 and Iba-1, for combined water-solubilized Coenzyme-10 and ashwagandha extract, according to exemplary embodiments of the application.

FIG. 4 shows A) images of immunofluorescent staining of the SN in midbrain sections probing for pro-survival neurotrophic factor GDNF (glial derived neurotrophic factor) and GFAP (glial fibrillary acidic protein), a marker of astroglia activation (scale bar=100 microns); and B) quantification of corrected total fluorescence for GDNF and GFAP, for combined water-solubilized Coenzyme-10 and ashwagandha extract, according to exemplary embodiments of the application.

FIG. 5 shows A) images of immunofluorescent staining at tip of the SN in midbrain sections probing for pro-survival neurotrophic factor pro-BDNF (brain derived neurotrophic factor) and tyrosine hydroxylase (scale bar=100 microns); and B) quantification of corrected total fluorescence for pro-BDNF and tyrosine hydroxylase, for combined water-solubilized Coenzyme-10 and ashwagandha extract, according to exemplary embodiments of the application.

FIG. 6 shows motor differences due to paraquat showing the proportion of rats with head up or head down on 5 cm rod rotating at 6 rpm, according to exemplary embodiments of the application.

FIG. 7 shows images of immunohistochemical staining for tyrosine hydroxylase (TH) indicating dopaminergic neurons in substantia nigra for water-solubilized ashwagandha extract (scale=25× and 100×), according to exemplary embodiments of the application.

FIG. 8 shows images of immunofluorescent staining at tip of the SN in midbrain sections probing for tyrosine hydroxylase (scale bar=100 microns); for combined water-solubilized ashwagandha extract, according to exemplary embodiments of the application.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least +5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “composition of the application” or “composition of the present application” and the like as used herein refers to a composition comprising one or more water-solubilized compounds of the application.

The term “suitable” as used herein means that the selection of the particular composition or conditions would depend on the specific steps to be performed, the identity of the components to be transformed and/or the specific use for the compositions, but the selection would be well within the skill of a person trained in the art.

The term “PTS monomer” as used herein refers to a compound having the following general structure:

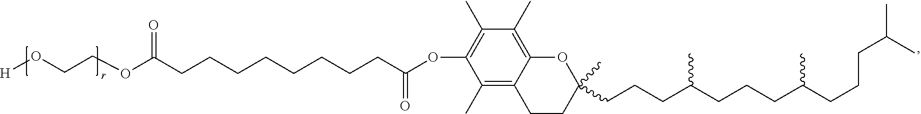

wherein r is 12 or 13.

The term “PTS dimer” as used herein refers to a compound having the following general structure:

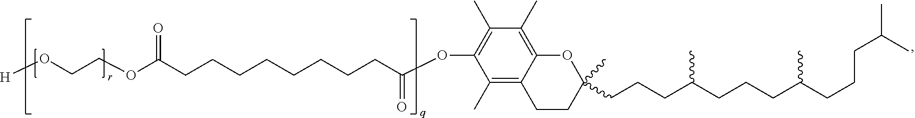

wherein r is 12 or 13; and q is 2.

The term “polyalcohol” as used herein refers a compound having the general formula HOCH₂(CHOH)ₓCH₂OH. In one embodiment, x is an integer between 1 and 2000, or 1 and 500, or 1 and 100, or 1 and 50 or 1 and 10, or 10 and 2000.

The term “polyether” as used herein refers to a compound that is an oligomer or polymer having repeating units comprising an ether functionality such as polyethylene glycol.

The present description refers to a number of chemical terms and abbreviations used by those skilled in the art. Nevertheless, definitions of selected terms are provided for clarity and consistency.

The term “aq.” as used herein refers to aqueous.

The term “PBS” as used herein refers to phosphate buffer saline.

The term “TBS” as used herein refers to tris-buffered saline.

The term “PQ” as used herein refers to paraquat.

The term “ASH” as used herein refers to ashwagandha root extract

The term “WS” as used herein refers to water-solubilized.

The term “Ubisol-Q10” as used herein refers to Coenzyme-Q10 water solubilized with PTS.

The term “TH” as used herein refers to tyrosine hydroxylase.

The term “DAB” as used herein refers to 3,3′-diaminobenzidine.

The term “DAPI” as used herein refers to 4′,6-diamidino-2-phenylindole.

The term “DA” as used herein refers to dopamine.

The term “SN” as used herein refers to substantia nigra.

The term “PD” as used herein refers to Parkinson's disease.

The term “AD” as used herein refers to Alzheimer's disease.

The term “MPTP” as used herein refers to 1-methyl-4-phenyl-1,2,3,6-tetrahydropyridine.

The term “ROS” as used herein refers to reactive oxygen species.

The term “cell” as used herein refers to a single cell or a plurality of cells and includes a cell either in a cell culture or in a subject.

The term “subject” as used herein includes all members of the animal kingdom including mammals, and suitably refers to humans.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition, or manifesting a symptom associated with a disease, disorder or condition.

The term “administered” as used herein means administration of a therapeutically effective amount of a compound, or one or more compounds, or a composition of the application to a cell either in cell culture or in a subject.

As used herein, the term “effective amount” or “therapeutically effective amount” means an amount of a compound, or one or more compounds, of the application that is effective, at dosages and for periods of time necessary to achieve the desired result.

The term “neurological disorder” as used herein refers to a disease, disorder or condition of the central and peripheral nervous system, i.e. the brain, spinal cord, cranial nerves, peripheral nerves, nerve roots, autonomic nervous system, neuromuscular junction, and muscles, characterized by structural, biochemical or electrical abnormalities in the nervous system. For example, more than 400 neurological disorders are listed by the National Institute of Neurological Disorders and Stroke such as, for example, Acute Spinal Cord Injury, Alzheimer's Disease, Amyotrophic Lateral Sclerosis (ALS), Ataxia, Bell's Palsy, Brain Tumors, Cerebral Aneurysm, Dementia, Epilepsy and Seizures, Guillain-Barré Syndrome, Headaches, Hydrocephalus, Meningitis, Multiple Sclerosis, Muscular Dystrophy, Neurocutaneous Syndromes, Parkinson's Disease, Strokes, Encephalitis, Septicemia.

The terms “neuroprotection” or “neuroprotective” as used herein refer to the relative preservation of neuronal structure and/or function. Neuroprotection aims to prevent or slow neurological diseases or disorders progression and secondary injuries by halting or at least slowing the loss of neurons. Common mechanisms behind neurodegeneration include increased levels in oxidative stress, mitochondrial dysfunction, excitotoxicity, inflammatory changes, iron accumulation, and protein aggregation.

As used herein, the term “effective amount” means an amount effective, at dosages and for periods of time, necessary to achieve a desired result.

The term “extract” is the result of a separation or isolation process of substances from a matrix or raw material. For example, extracts are obtained from the separation of certain desired components from the whole or part of a plant, such as its leaves, flowers, fruits, peel, bark, etc.

The term “bioavailability” as used herein refers to the proportion of a drug or other substance which enters the circulation (bloodstream) when introduced into the body and so is able to have an active effect.

### II. Compositions of the Application

It has been surprisingly shown herein that formulations of the present application provide neuroprotective properties. The formulations of the present application including water-solubilized compounds further provide for increased bioavailability of the compounds. Comparable formulations did not display the same properties, highlighting the surprising results obtained with the water-solubilized formulations of the application.

Accordingly, the present application includes a formulation comprising:
(i) Coenzyme-Q10 water-solubilized with at least one solubilizing agent of Formula (I):

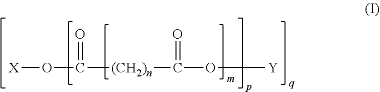

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
(ii) an alcohol-extracted ashwagandha root extract.

The present application further provides a formulation comprising:
(i) an alcohol-extracted ashwagandha root extract water-solubilized with at least one solubilizing agent of Formula (I):

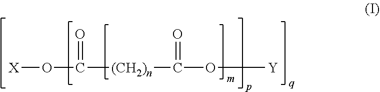

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2.

The present application further provides a formulation comprising:
(i) Coenzyme-Q10 water-solubilized with at least one solubilizing agent of Formula (I):

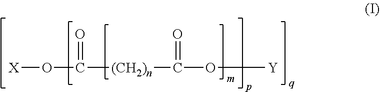

wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
(ii) an alcohol-extracted ashwagandha root extract water-solubilized with at least one solubilizing agent of Formula (I).

In some embodiments, the hydrophobic moiety in Formula (I) is selected from cholesterol, 7-dehydrocholesterol, campesterol, sitosterol, ergosterol, stigmasterol, α-tocopherol, β-tocopherol, γ-tocopherol and δ-tocopherol. In some embodiments, the hydrophobic moiety is α-tocopherol.

In some embodiments, the hydrophilic moiety in Formula (I) is a polyether. In some embodiments, the polyether is a polyalkylene glycol. In some embodiments, the polyalkylene glycol is a polyethylene glycol or a polypropylene glycol. The term “polyalkylene glycol” as used herein includes polyalkylene glycols having an esterifiable hydroxy group at least at one end of the polymer as well as derivatives of such polymers having esterifiable carboxy groups. The residue of the hydrophilic moiety is the entire hydrophilic molecule, except for its esterified hydroxy or carboxy group or groups, such as a terminal hydroxy group of a polyethylene glycol. In some embodiments, the polyethylene glycol has an average molecular weight of from about 300 to about 5000, or about 400 to about 1000.

In some embodiments, the hydrophilic moiety in Formula (I) is a polyalcohol.

In some embodiments, when p, m and q are all equal to 1 and the hydrophobic moiety is cholesterol, n is greater than 4 and not equal to 8.

In some embodiments, when p, m and q are all equal to 1 and the hydrophobic moiety is α-(+)-tocopherol, n is not equal to 2.

In some embodiments, n is an integer of from 2 to 10, or 6 to 10, or 8.

In some embodiments, the at least one compound of Formula (I) is polyoxyethanyl-α-tocopheryl sebacate (PTS) monomer, PTS dimer or combinations thereof. In some embodiments, the at least one compound of Formula (I) is PTS monomer. In some embodiments, the at least one compound of Formula (I) is PTS dimer. In some embodiments, the at least one compound of Formula (I) is a combination of PTS monomer and PTS dimer.

In some embodiments, the ratio of the solubilizing agent to the Coenzyme-Q10 (w/w) is from about 2:1 to about 12:1 or about 2:1 to 5:1, or about 2:1 or about 3:1. In some embodiments, the ratio of the water-solubilized Coenzyme-Q10 to the ashwagandha root extract is from about 1:20 to about 1:60, or from about 1:30 to about 1:50, or about 1:40. In some embodiments, the ratio of the solubilizing agent to the ashwagandha root extract (w/w) is from about 10:1 to about 1:1, or from about 5:1 to 2:1, or about 3:1 or about 2.8:1. In some embodiments, the ratio of the water-solubilized Coenzyme-Q10 to the water-solubilized ashwagandha root extract is from about 1:40 to about 1:5, or from about 1:30 to about 1:10, or about 1:20.

In some embodiments, the formulation contains from about 0.1 mg/mL to about 100 mg/ml of water solubilized Coenzyme-Q10. In some embodiments, the formulation contains from about 25 mg/ml to about 75 mg/ml of water solubilized Coenzyme-Q10. In some embodiments, the formulation contains about 50 mg/ml of water solubilized Coenzyme-Q10. In some embodiments, the formulation contains from about 0.1 μg/mL to about 100 μg/ml of water solubilized Coenzyme-Q10, after dilution for administration. In some embodiments, the formulation contains from about 25 μg/mL to about 75 μg/ml of water solubilized Coenzyme-Q10, after dilution for administration. In some embodiments, the formulation contains about 50 μg/mL of water solubilized Coenzyme-Q10, after dilution for administration.

In some embodiments, the formulation contains from about 0.1 mg/mL to about 300 mg/ml of the ashwagandha root extract. In some embodiments, the formulation contains from about 150 mg/mL to about 250 mg/ml of the ashwagandha root extract. In some embodiments, the formulation contains about 200 mg/mL of the ashwagandha root extract. In some embodiments, the formulation contains from about 0.1 mg/mL to about 3 mg/mL of the ashwagandha root extract, after dilution for administration. In some embodiments, the formulation contains from about 1.5 mg/mL to about 2.5 mg/ml of the ashwagandha root extract, after dilution for administration. In some embodiments, the formulation contains about 2 mg/mL of the ashwagandha root extract, after dilution for administration.

In some embodiments, the formulation contains from about 0.1 mg/mL to about 20 mg/ml of the water-solubilized ashwagandha root extract. In some embodiments, the formulation contains from about 1 mg/mL to about 15 mg/ml of the water-solubilized ashwagandha root extract. In some embodiments, the formulation contains about 10 mg/mL of the water-solubilized ashwagandha root extract. In some embodiments, the formulation contains from about 0.5 mg/mL to about 3 mg/mL of the water-solubilized ashwagandha root extract, after dilution for administration. In some embodiments, the formulation contains from about 0.75 mg/mL to about 2 mg/ml of the water-solubilized ashwagandha root extract, after dilution for administration. In some embodiments, the formulation contains about 1 mg/mL of the water-solubilized ashwagandha root extract, after dilution for administration.

In some embodiments, extraction from ashwagandha root is conducted according to known extraction technique in the art. In some embodiments, the extract is an alcohol-extracted ashwagandha root extract. In some embodiments, the alcohol-extracted ashwagandha root extract is ethanol-extracted ashwagandha root extract. Ashwagandha contains various alkaloids, steroidal lactones and saponins, which may be extracted according to known methods. Without being bound to theory, some compounds thought to be the main active components of ashwagandha include withanolides and sitoindosides. These compounds are alleged to have antistress/adaptogenic, antitumor, tonic, anxiolytic, anti-inflammatory and antiarthritic properties. It will be appreciated that ethanol may be used as the extraction solvent due to its low toxicity, and ability to extract hydrophobic phytochemicals effectively. Other solvents may be used, for example: other alcohols such as methanol or other low molecular weight alcohols (such as butanol, pentanol), or chloroform, hexanes, etc. and this is well within the purview of a skilled person.

Also provided is an emulsion comprising a formulation of the present application dispersed in water. It will be appreciated by a person skilled in the art that embodiments relating to the compositions in the emulsions of the present disclosure can be varied as described herein in relation to the compositions of the present disclosure. In some embodiments, the formulation is dispersed in the water in the form of micelles. In some embodiments, the micelles have an average size of less than about 50 nm. In some embodiments, the emulsion further comprises adjuvants, colorants, flavoring agents, preservatives, buffers and combinations thereof.

The present disclosure also includes a pharmaceutical or cosmetic formulation comprising an emulsion of the present disclosure and a biologically acceptable carrier. The present disclosure further includes a dietary supplement comprising an emulsion of the present disclosure and a biologically acceptable carrier. The emulsions of the present disclosure may advantageously provide a format for subjects that does not involve swallowing pills but instead offers a solution for the administration or use in an aqueous emulsion format or cream. Accordingly, the present disclosure also includes a pharmaceutical or cosmetic formulation comprising an emulsion of the present disclosure and a biologically acceptable liquid carrier as well as a dietary supplement comprising an emulsion of the present disclosure and a biologically acceptable liquid carrier.

In some embodiments, the pharmaceutical or cosmetic formulation is in the form of a spray, syrup or drop. In another embodiment of the present disclosure, the dietary supplement is in the form of a spray, syrup or drop. A person skilled in the art would know how to prepare suitable formulations.

The present application also includes a beverage comprising the emulsion of the present application.

The present application also includes a use of an emulsion of the present application for the preparation of a pharmaceutical formulation or a cosmetic formulation. The present application further includes a use of an emulsion of the present application for the preparation of a dietary supplement. The present application also includes a use of an emulsion of the present application for the preparation of a beverage. For example, an emulsion of the present application can be added to any suitable beverage base such as water. In an embodiment, from about 1 mL to about 3 mL or about 2 mL of the emulsion is added per every 250 mL of the beverage base such as water.

### III. Methods and Uses of the Application

The formulations of the application have shown neuroprotective properties.

Accordingly, the present application includes a method for treating, reducing or preventing a neurological disease or disorder in a subject in need thereof, comprising administering a pharmaceutically effective amount of a formulation of the application to the subject.

In some embodiments, the neurological disease or disorder is selected form Alzheimer's disease, Parkinson's disease, neuroinflammation, oxidative stress, mitochondrial dysfunction, dementia and autophagy/proteosome deficiency.

In some embodiments, the neurological disease or disorder involves at least one biochemical mechanism selected from oxidative stress, mitochondrial impairment, autophagy/proteosome impairment, and elevated neuroinflammation.

The present application further provides use of formulations of the present application for the treatment, reduction or prevention of a neurological disease or disorder.

The present application further includes use of formulations of the present application in the manufacture of a medicament for the treatment, reduction or prevention of a neurological disease or disorder.

### IV. Methods of Preparing the Compounds and Compositions of the Application

In some embodiments, the solubilizing agent of Formula (I), for example solubilizing agent PTS, of the present application are prepared by mixing a solution of α-tocopherol (T) and triethylamine in a suitable solvent and slowly adding the mixture to a solution of sebacoyl chloride (S) in a suitable solvent. This reaction mixture is added slowly to a solution of PEG 600 (P) and triethylamine in a suitable solvent, then subsequently subjected to aqueous washes, and organic washes. Any methods known in the art may alternatively used, and this is well within the purview of a skilled person in the art.

In some embodiments, the formulations of the present application are prepared by heating a formulation comprising Coenzyme-Q10 or ashwagandha root extract and a compound of Formula (I) (for example, the solubilizing agent PTS) to form a homogeneous melt.

In some embodiments, the water-soluble formulations are used to form stable emulsions having a micelle size of less than about 50 nm, or between about 7.5 to about 40.0 nm or between about 10 to about 30 nm, and are prepared by heating a formulation comprising Coenzyme-Q10 or ashwagandha root extract and a compound of Formula (I) (for example, the solubilizing agent PTS) to form a homogeneous melt and combining the homogeneous melt with water using either blending or high shear mixing, optional homogenization by use of a microfluidizer and rapid cool down using cooling, ice, cold water, or a mixture of ice and water to obtain the emulsion.

Accordingly, the present application also includes a method for preparing an emulsion, the method comprising:
heating a formulation of the present application to form a homogeneous melt; and
combining the homogeneous melt with water to obtain the emulsion.

It will be appreciated by a person skilled in the art that embodiments relating to the compositions in the methods for preparing an emulsion of the present application can be varied as described herein in relation to the compositions of the present application.

In an embodiment, the combining step comprises mixing the homogeneous melt and water at a temperature of from about 40° C. to about 95° C., for example, a temperature of from about 75° C. to about 80ºC for a time of about 15 minutes to about 4 hours, or about 15 minutes to about 60 minutes, or about 30 minutes.

In one embodiment, the combining step comprises mixing the homogeneous melt with water using either blending or high shear mixing, optionally followed by homogenization by use of a microfluidizer, and rapid cool down, using cooling, ice, cold water, or a mixture of ice and water to obtain the emulsion

In some embodiments, subsequent to mixing, the method further comprises processing the mixture through a microfluidizer. The conditions for processing the mixture through a microfluidizer are any suitable conditions. In an embodiment, the conditions comprise a single pass through the microfluidizer. In another embodiment, the conditions comprise a pressure of from about 10,000 psi to about 20,000 psi.

In another embodiment, the mixture is passed through a filter having a pore size of about 0.2 um.

In an embodiment, the method further comprises cooling the mixture. It will be appreciated by the person skilled in the art that in embodiments comprising processing the mixture through a microfluidizer, the cooling can be subsequent or simultaneous to the processing of the mixture through the microfluidizer. The mixture is cooled to any suitable temperature. In an embodiment, the mixture is cooled to a temperature of about 1° C. to about 15° C. or about 4° C. In one embodiment, the mixture is cooled using ice.

In some embodiments, the cooling comprises mixing the homogeneous emulsion with ice or a combination of water and ice. In an embodiment, the ratio by volume of water:ice in the final combination is about 2:1. In other embodiments, the mixture is cooled using cooling systems.

### EXAMPLES

The following non-limiting examples are illustrative of the present application. As is apparent to those skilled in the art, many of the details of the examples may be changed while still practicing the disclosure described herein.

### General Methods

### Ethanolic Extraction of Ashwagandha Root

Ashwagandha root powder (Premier Herbal Inc., ON, Canada) was soaked/stirred in anhydrous ethanol at a ratio of 1:10(w/v) at ˜70° C. for 24 hrs. Following 24 hrs, the crude extract was filtered through a P8 paper filter and ethanol removed using a rotary evaporator. The solid extract was then resuspended with anhydrous ethanol to a concentration of 200 mg/mL.

### Animal Care

All animal care, treatments, and procedures were approved by the University of Windsor's Animal Care Committee in accordance with the Canadian Council for Animal Care guidelines. Experiments were conducted on male Long Evans Hooded rats (Charles River Laboratories). Rats arrived at 2.5 months of age and were habituated/trained for behavioural testing until an age of 5 months. Rats were housed in groups of 3-4 animals/cage for convenience and to prevent hierarchies that could arise due to extent of neurodegeneration. Rats had independent feeding schedules to pre-vent competition. Animals were housed at 20° C. under a 12-hr dark-light cycle to ensure they were awake during the day for behavioural assessments.

### Injection Regimen

Rats underwent the injection regimen at 5 months of age. Rats received 5 intraperitoneal injections of PQ dissolved in 1× phosphate buffered saline (PBS) at 10 mg/kg body weight/injection. One injection occurred every 5 days over 20 days. Control rats received only saline injections according to the same schedule as PQ injected rats.

### Drinking Water Treatment

Animals were provided the following treatments in their drinking water 24 hrs after the last day of injections: Saline injected rats given plain drinking water (n=7); saline injected rats given the tonic (combination of 50 μg/mL Ubisol-Q10 (water-solubilized Coenzyme-Q10 provided by Next Remedies, Toronto, ON, Canada) and 2 mg/mL ethanolic ashwagandha extract (ASH))(n=5); PQ inject rats given plain drinking water (n=9); PQ injected rats given PTS carrier (n=6); PQ injected rats given 50 μg/mL Ubisol-Q10 (n=7); PQ injected rats given 2 mg/mL ASH (n=8); PQ injected rats given the tonic (n=8). Groups where ASH was not provided had 1% anhydrous ethanol added to the drinking water to account for 1% ethanol present in water when ASH was added. Fresh drinking solutions were provided every 3-4 days. Treatment continued for 4 months during which behavioural assessments were conducted. Following 4 months, animals were sacrificed, and their brains extracted for biochemical analysis.

### Behavioural Assessments

Motor/balance coordination on the rotorod. The rats' ability to maintain balance on a slowly rotating cylinder was measured with a rotorod apparatus similar to that previously described. This apparatus consists of a 15×7 cm sandpaper-covered (80 grid) wooden dowel attached to a variable speed motor. Clockwise revolutions of the rotorod can be adjusted from 6 to 12 R.P.M. The rod is separated from the motor by a vertical 30×48 cm grey wooden panel that is scored with black vertical and horizontal lines to form 12×12 cm squares. The apparatus rests on a small table so that the dowel is 27 cm above its surface. A digital video camera is positioned 1 m in front of and level with the rod. Regular fluorescent ceiling lighting and a 60-W lamp approximately 3 m in front of the apparatus illuminates it. The mpeg recordings of each rat's rotorod performance was converted to jpeg images at a rate of 5 frames per second. We analyzed each animal's movements over its last 500 frames (100 seconds). The position of the tip of the nose was tracked on a 450×450 pixel Cartesian system of coordinates with tracking software (Seven Software, Inc, Montana, U.S.A.). The grid is divided by a horizontal line above the rotorod and by two vertical lines, one to the right and the other to the left. The proportion of frames in which the animal's nose is beyond the left and beyond the right of these vertical lines is our measure of proportion of time the animal spends walking forward and backward, respectively. We note from our past experience that the time the animal's nose is between the two vertical lines, as it is when turning around, does not account for more than 10% of its total time on the rotorod.

### Tissue Preparation for Immunohistochemistry

Following the experimental period, rats were euthanized and perfused with ice-cold PBS containing 28 ug/mL heparin (Sigma-Aldrich, Canada, Cat. No. H3393) followed by fixation with ice-cold 10% formaldehyde made in PBS. Following perfusion, brains were stored in 10% formalin at 4° C. To prepare for sectioning, brains were incubated in 30% sucrose (w/v in PBS) until brains sank in the solution. Following sucrose incubation, brains were cryosectioned at 30 μm thickness with Shandon™ M-1 embedding matrix (Thermo Scientific Canada, Cat. No. 1310TS) onto glass microscope slides.

### Immunohistochemistry (Colourimetric)

Sections were washed for 5 min twice with tris-buffered saline (TBS), followed by incubation with 0.3% H₂O₂ to block endogenous peroxidase activity. Sections were rinsed for 5 min twice with TBS, followed by a 30 min block with DAKO™ serum-free protein block (Agilent Technologies Canada Inc., Cat. No. X0909) and normal serum according to instructions of the Vector Laboratories Vectastain™ Elite ABC-Peroxidase kit, rabbit IgG (MJS BioLynx Inc., Cat. No. VECTPK4001). Tissue sections were incubated overnight at 4° C., with tyrosine hydroxylase (TH) primary antibody (rabbit IgG; 1:1000; cat. no. P40101-150) (Pel-Freeze Biologicals, USA). Tissue sections were washed for 5 min twice with TBS, followed by incubation with secondary biotinylated antibody according to instructions from the Vectastain Elite ABC Peroxidase kit. Sections were washed twice with TBS for 5 min, then incubated with avidin-conjugated horseradish peroxidase from the Vectastain Elite ABC-Peroxidase kit for 45 min. Sections were washed twice with TBS for 5 min and incubated with 3,3′-diaminobenzidine (DAB) stain solution according to the Vector Laboratories DAB peroxidase substrate kit (MJS BioLynx Inc., Cat. No. SK-4100). Sections were dehydrated with two 5 min washes in anhydrous ethanol then a 7 min xylenes wash followed by cover slipping using Per-mount® mounting medium (Fisher Scientific Canada, Cat. No. SP15-500). Cells were imaged using bright-field microscopy via a Leica DMI6000 B inverted microscope (Leica Microsystems, Concord, ON, Canada).

### Immunohistochemistry (Fluorescent)

Sections were washed for 5 min twice with TBS, followed by incubation with DAKO serum-free protein block (Agilent Technologies Canada Inc., Cat. No. X0909). Tissue sections were then incubated overnight at 4° C. in the following primary antibodies: glial fibrillary acidic protein (GFAP) (rabbit IgG, 1:500; Novus Biologicals, cat. no. NB300-141), (Iba-1) (rabbit IgG, 1:300; Novus Biologicals, cat. no. NB100-1028), tyro-sine hydroxylase (rabbit IgG, 1:1000; Pel-Freeze Biologicals, cat. no. P40101-150), be-clin-1 (mouse IgG, 1:500; Santa Cruz Biotechnology, cat. no. sc-48342), pro-BDNF (mouse IgG, 1:500; Santa Cruz Biotechnology, cat. no. sc-65513), GDNF (mouse IgG, 1:500; Santa Cruz Biotechnology, cat. no. sc-13147), 4-hydroxynonenal (rabbit IgG, 1:500; Abcam Inc., cat. no. ab46545), CARP1 (rabbit IgG, 1:1000; provided by Dr. Arun Rishi of Wayne State University). The following day, tissue sections were washed for 5 min twice with TBS and incubated at room temperature for 2 hrs in the following secondary antibodies: Vector Laboratories fluorescein horse anti-mouse IgG (1:500; MJS BioLynx Inc., Cat. No. FI-2000), and Alexa Fluor™ 568 goat anti-rabbit IgG (Thermo Scientific Canada, Cat. No. A11011). Sections were then washed twice for 5 min in TBS followed by cover slipping with Vectashield® Vibrance™ antifade mounting medium with DAPI (MJS BioLynx Inc., Cat. No. VECTH18002). Tissue sections were imaged using epifluorescence microscopy via a Leica DMI6000 B inverted microscope (Leica Microsystems, Concord, ON, Canada). Fluorescence was quantified in images captured using ImageJ software.

### Example 1—Water-Solubilized Coenzyme-Q10+Ashwagandha Root Extract

### 1.1 Water-Solubilized Coenzyme-Q10 and Ashwagandha Combined Are More Effective at Protecting DA Neurons in SN From PQ Toxicity Compared to the Reagents Alone

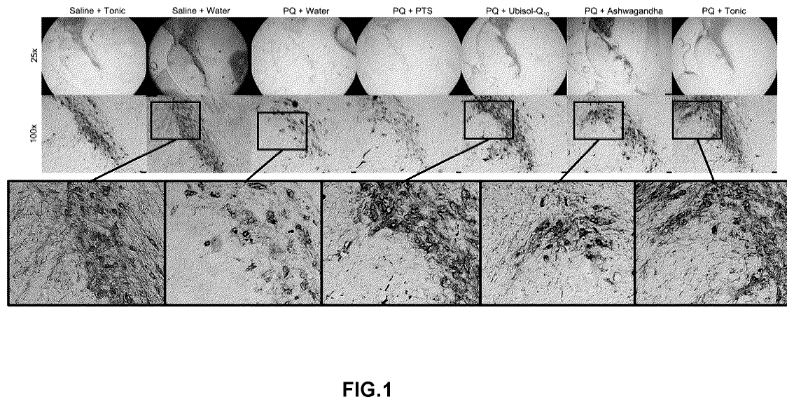

FIG. 1 shows that combined formulations of water-solubilized Coenzyme-Q10 and ashwagandha extract halt progression of neurodegeneration of substantia nigra neurons and thus protect dopaminergic neurons better than reagents alone. Immunohistochemical staining for tyrosine hydroxylase (TH) indicating dopaminergic neurons in substantia nigra are shown. Rats were injected with saline as a control and given either plain drinking water or water supplemented with combined Ubisol-Q10 (2 mg/kg/day) and ethanolic ashwagandha extract (120 mg/kg/day) (Tonic). Rats were also injected with paraquat (PQ) to stimulate Parkinsonism and were given plain water, or either water supplemented with PTS, Ubisol-Q10 (2 mg/kg/day), ethanolic ashwagandha extract (120 mg/kg/day), or the combination of Ubisol-Q10 and ashwagandha extract (combo). The combo treatment (green circle) was more effective than Ubisol-Q10 (yellow circle) or ashwagandha extract (red circle) alone as indicated by greater TH+ immunoreactivity (staining) and greater protection of neuron morphology (greater number of fibers extending from cell bodies).

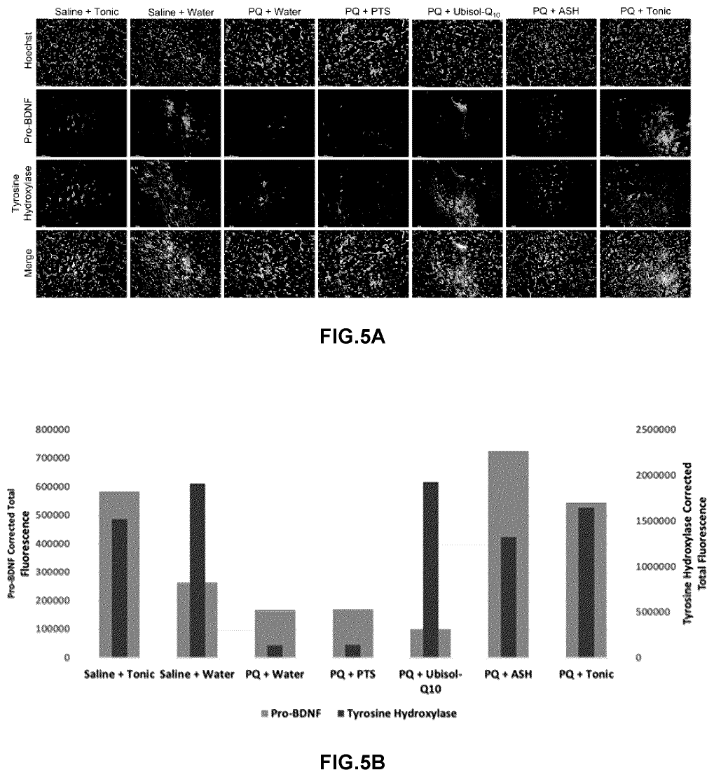

Previously it was shown that Ubisol-Q10 protects DA neurons in rat/mouse brains after exposure to PQ/MPTP. Similarly, ashwagandha extract (ASH) was shown to protect mouse brains post exposure to MPTP. Here, Ubisol-Q10 and ASH were combined to examine if the reagents combined are more effective compared to them used alone. Confirming past results, it was indeed observed that rats exposed to PQ and given only plain drinking water, or the PTS vehicle had significant neurodegeneration in the SN as indicated by significantly reduced immunoreactivity for tyrosine hydroxylase (TH) (a marker of DA neurons) (FIG. 1). Also confirming previous results with Ubisol-Q10 or ASH, rats exposed to PQ and given either Ubisol-Q10 or ASH alone had significant protection of their DA neurons (FIG. 1). Interestingly, while ASH was not as effective as Ubisol-Q10 in protecting DA neurons, the morphology of the neurons was better maintained with ASH treatment as there were a greater abundance of fibers extending from the cell bodies appearing more similar to neurons in the animal injected with only saline and given plain drinking water (FIG. 1). When PQ treated rats were given the tonic treatment (combination of Ubisol-Q10 and ASH), the benefits of both treatments were noted in that Ubisol-Q10 maintains the number of DA neurons while ASH maintained their morphology. Indeed, the PQ+tonic rats' SNs appeared almost identical to the control (saline+water) group (FIG. 1). Furthermore, it was desirable to confirm that the tonic treatment of Ubisol-Q10 and ashwagandha was not toxic to healthy animal brains. Indeed, the tonic treatment did not result in any observable neurodegeneration of the SN in animals injected with saline (FIG. 1). Along with colourimetric immunohistochemistry to detect TH, immunofluorescent staining (FIG. 5) was also performed. While immunofluorescent images were taken to-wards to the tip of the SN instead of the overall/center of the SN as in FIG. 1, similar observations were made in the rats' brains. PQ treated animals given plain water or PTS had significantly reduced levels of fluorescence for TH compared to the two saline injected groups. Treatment with Ubisol-Q10, ASH, or the tonic had significantly higher amounts of TH fluorescence compared to PQ animals fed plain water or PTS.

### 1.2 Water-Solubilized Coenzyme-Q10 and ASH Act as Potent Antioxidants Against PQ Induced Neurotoxicity

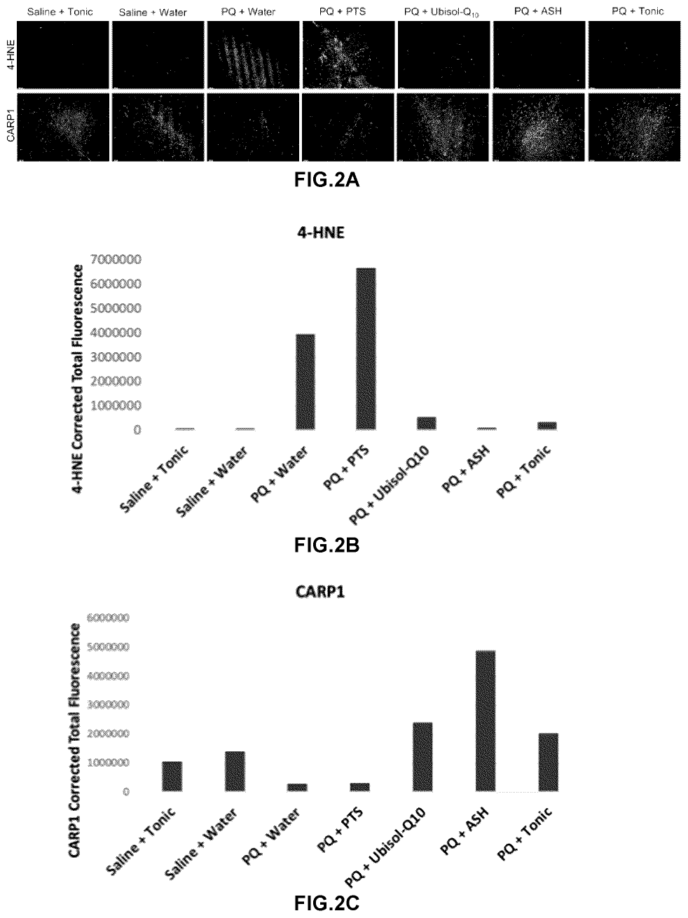

FIG. 2 shows that Ubisol-Q10 and ashwagandha reduces paraquat induced lipid peroxidation and enhance expression of CARP1 in SN of rat brains. Immunofluorescent staining of the SN in midbrain sections probing for oxidative stress marker and lipid peroxidation by-product 4-hydroxynonenal (4-HNE) and apoptosis regulator CARP1 are shown. 4-HNE is significantly elevated in PQ treated rats given only plain drinking water or PTS vehicle. 4-HNE is drastically reduced in Saline control groups and PQ treated rats given Ubisol-Q10, ashwagandha or the tonic of both. CARP1 is upregulated in PQ treated animals given Ubisol-Q10, ashwagandha or the tonic of both similar to the two saline control groups. There was minimal immunoreactivity of CARP1 in PQ treated rats given plain drinking water or PTS vehicle.

It is well known that exposure to PQ results in increased production of ROS. Confirming results from these other studies, significant increases in levels of lipid peroxidation product, 4-hydroxynonenal (4-HNE), were observed in the brains of PQ treated rats given plain or PTS supplemented drinking water (FIG. 2A, 2B). When these animals were given Ubisol-Q10, ASH, or the tonic, the pro-oxidant effects of PQ were almost entirely reversed appearing similar to the saline injected rats given plain water or the tonic (FIG. 2A, 2B).

It can also be seen that apoptosis regulator CARP1 expression is significantly reduced with PQ treatment. Cell division cycle and apoptosis regulator 1 (CAPR1) as its names implies is involved with regulating cell death. CARP1 is known to be a positive regulator of apoptosis. The status of CARP1 and its role in PQ mediated neurotoxicity were investigated. Interestingly, CARP1 levels were significantly reduced in the SN of PQ injected rats fed plain water or PTS compared to the saline groups (FIG. 2A, 2C). Furthermore, PQ injected rats fed Ubisol-Q10, ASH, or the tonic had higher levels of CARP1 compared to the saline groups (FIG. 2A, 2C).

### 1.3 Autophagy Regulator Beclin-1 Is Upregulated in Rats Fed Water-Solubilized Coenzyme-Q10

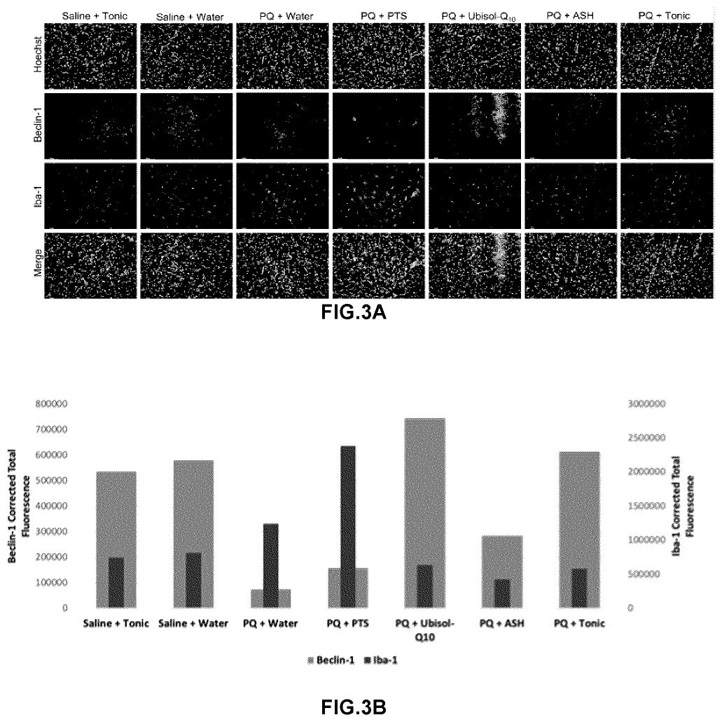

FIG. 3 shows that Ubisol-Q10 and ashwagandha inhibit activation of pro-inflammatory microglia and stimulate activation of autophagy. Immunofluorescent staining of the SN in midbrain sections probing for belin-1, a major regulator of autophagy and Iba-1 (ionized calcium-binding adapter molecule 1), a marker of microglia activation. B) quantification of respective fluorescence are shown. Microglia activation is inhibited in PQ treated rats given Ubisol-Q10 and ashwagandha as indicated by the reduced amount of red ameboid shaped microglia cells/reduced iba-1 immunoreactivity. Rats given Ubisol-Q10 showed significant increased expression of beclin-1, a major regulator of autophagy. Ashwagandha treated rats also had increased expression of beclin-1 to a lesser degree than Ubisol-Q10. Nuclei were counterstained with DAPI.

It was shown that when AD fibroblast and transgenic AD mice were treated with Ubisol-Q10, major autophagy regulator beclin-1 was upregulated compared to untreated groups. As mentioned earlier, PD and AD share several biochemical mechanisms leading to neurodegeneration which include impaired autophagy. It was previously found that autophagy resumption via belcin-1 was a conserved mechanism in both human AD fibroblasts and the brains of transgenic AD mice. Investigations were conducted to confirm if this same mechanism was conserved in the rats of this study. Indeed, animals injected with PQ and fed plain or PTS supplemented water has significantly decreased levels of beclin-1 compared to saline injected animals (FIG. 3). Similar to previous studies, beclin-1 expression was increased in rats given Ubisol-Q10 (FIG. 3). Interestingly, while not as effective of Ubisol-Q10, PQ inject rats fed ASH also had elevated levels of beclin-1 compared to PQ rats fed plain water or PTS (FIG. 3).

### 1.4 Water-Solubilized Coenzyme-Q10 and ASH Inhibit Pro-Inflammatory Microglia and Activate Pro-Survival Astroglia

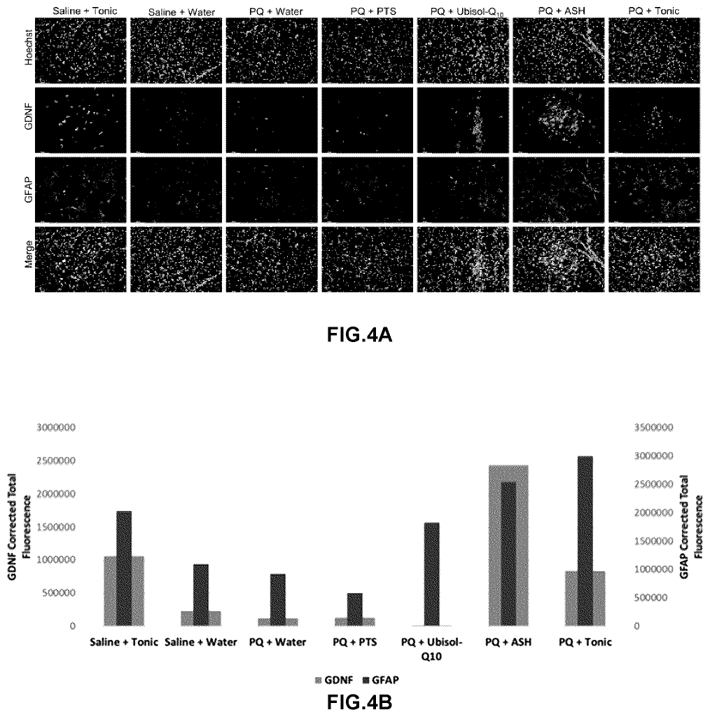

FIG. 4 shows that Ubisol-Q10 and ashwagandha extract enhance activation of pro-survival astroglia. Immunofluorescent staining of the SN in midbrain sections probing for pro-survival neurotrophic factor GDNF (glial derived neurotrophic factor) and GFAP (glial fibrillary acidic protein), a marker of astroglia activation and quantification of respective fluorescence are shown. Astroglia activation is enhanced in PQ treated rats given Ubisol-Q10 and ashwagandha as indicated by the greater GFAP immunoreactivity/greater number of red fibers extending off the astroglia cell bodies. Only rats given ashwagandha showed increased expression of GDNF. Nuclei were counterstained with DAPI.

As mentioned previously, it has been observed in PD patients that pro-inflammatory microglia are active and pro-survival astroglia activity is reduced. Ashwagandha was shown to target inflammation and reduce oxidative stress in both AD and PD rodent models. The status of both microglia and astroglia were examined (FIG. 3, 4). Indeed, in rats injected with PQ and only fed plain water or PTS, levels of pro-inflammatory microglia were elevated compared to saline injected animals (FIG. 3). Similar to previous studies, it was observed that presence of active pro-survival astroglia was reduced in PQ treated rats fed plain water or PTS. Interestingly, with either treatment of Ubisol-Q10, ASH, or the tonic, microglia activation was significantly reduced compared to PQ treated rats given plain water or PTS. Further-more, microglia activation was even reduced slightly in saline injected animal given the tonic compared to the saline animals given water. Compared to microglia, an opposite trend was observed with astroglia activation in FIG. 4. Animals injected with PQ and fed plain water or PTS had reduced activation of astroglia compared to saline injected animals. Ubisol-Q10 or ASH fed PQ treated animals showed significant in-creases in activation of astroglia compared to the PQ animals fed plain water or PTS. Furthermore, when combined, Ubisol-Q10 and ASH had an even greater effect on astroglia activation compared to reagents alone (FIG. 4). Interestingly, the saline injected animals given the tonic had around double the amount of astroglia activation compared to saline animals given plain water.

### 1.5 Levels of Neurotrophic Factors, GDNF and Pro-BDNF Are Elevated in Rats Fed ASH

FIG. 5 shows that pro-BDNF is upregulated in TH positive neurons of rats given ashwagandha but not Ubisol-Q10. Immunofluorescent staining at tip of SN in midbrain sections probing for pro-survival neurotrophic factor pro-BDNF (brain derived neurotrophic factor) and tyrosine hydroxylase and quantification of respective fluorescent are shown. Only TH neurons of rats given ashwagandha showed increased immunoreactivity for pro-BDNF. Nuclei were counterstained with DAPI.

It has been postulated that astrocytes secrete several pro-survival neurotrophic factors including BDNF and GDNF. Along with looking at the status of astrocytes, we also probed for both GDNF and pro-BDNF to examine if neurotrophic factor levels are affected by astrocyte activity. Interestingly, all animal groups that contained ASH in their drinking water had significantly elevated levels of GDNF compared to all other groups (FIG. 4). Similarly, pro-BDNF was also significantly elevated in all groups with drinking water containing ASH compared to groups without ASH (FIG. 5).

### 1.6 PQ Induced Motor Deficits Are Reduced in Rats Fed Ubisol-Q10 and ASH

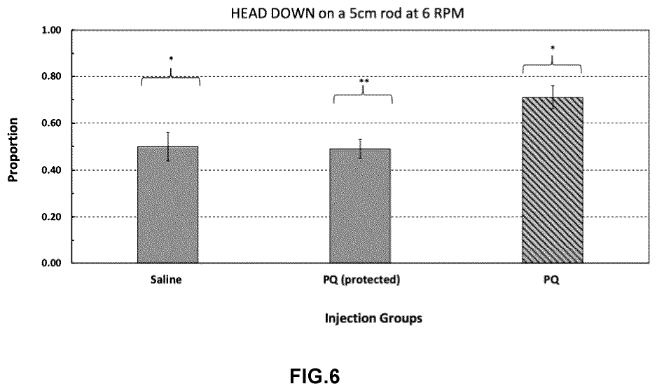

FIG. 6 shows that Ubisol-Q10 and ashwagandha reduce motor differences due to paraquat. The proportion of a rat with head up or head down was measured on 5 cm rod rotating at 6 rpm. Rats given saline injections and rats given paraquat injections who were fed the tonic (protected group) in their drinking water had their head down less so on the 5 cm rod compared to rats given paraquat injections who received plain drinking water. * p<0.05; ** p<0.01

Chronic PQ exposure in rats is known to cause motor impairments. Seen in FIG. 6, rats injected with PQ and given plain water (right) spent a greater proportion of their head down on the rotorod compared to saline injected animals fed plain water (left). Appearing similar to saline injected rat, animals injected with PQ and fed the tonic (center) showed a reduced proportion of their head down compared the PQ injected rats fed plain water.

### Discussion

In this study, Ubisol-Q10 and ethanolic ashwagandha extract (ASH) combined, two simple and well tolerated nutraceuticals, contributed in a complimentary way to target the multiple biochemical mechanisms implicated in PD. PD being a multifactorial disease, the two reagents combined were more effective at halting the progressive neurodegeneration of PD compared to the reagents alone. Furthermore, by combining the reagents, it was possible to use lowered doses compared to other studies as the reagents are able to target different mechanisms of PD. For the first time, a water-soluble formulation of coenzyme-Q10, Ubisol-Q10, was shown to induce autophagy via activation of beclin-1. Previously, this mechanism of autophagy induction via Ubisol-Q10 was observed in in-vitro and in-vivo models of AD. Along with autophagy induction, both Ubisol-Q10 and ASH acted as potent antioxidants against the pro-oxidant effects of PQ, reduced activation of pro-inflammatory microglia, and stimulated activation of pro-survival astroglia. Interestingly, ASH also resulted in secretion of neurotrophic factors, BDNF and GDNF.

Paraquat is an herbicide and environmental toxin well known to cause people to develop Parkinson's Disease when exposed. Other mammals such as rats are also known to develop similar neurodegeneration in the SN when exposed to PQ. The toxic effects of PQ have been studied in rats, and a well-established model has been developed. After chronic exposure to low doses of PQ, progressive neurodegeneration of DA neurons occurs in the brains of rats. As mentioned by Muthukumaran et al., BMC Neurosci. 2014. doi: 10.1186/1471-2202-15-21, following the final injection of PQ (after a series of 5 injections of 10 mg/kg/injection every 5 days), around 20% of DA neurons in the SN will have died. Further neurodegeneration occurs in the following weeks. Ubisol-Q10 has been shown to protect cultured neurons and DA neurons in the brains of rats against the toxic effects of PQ and other toxins such as MPTP. Furthermore, it's been shown that Ubisol-Q10 is able to target some of the biochemical mechanisms of PD such as oxidative stress, mitochondrial dysfunction, and autophagy. While Ubisol-Q10 did target some mechanisms of PD, it did not target all. With PD being a multifactorial disease, targeting only one or a few mechanisms is not enough to halt neurodegeneration. It was considered to combine Ubisol-Q10 with another reagent, one that may target the other mechanisms of PD that Ubisol-Q10 did not such as inflammation. Mentioned previously, ashwagandha extracts have shown to reduce DA neuron death in rodent models of PD by targeting oxidative stress and neuroinflammation. As a result, Ubisol-Q10 was combined with ethanolic extract of ashwagandha and indeed, showed that DA neurons were better protected compared to the reagents alone (FIG. 1). While Ubisol-Q10 was able to maintain the overall amount of DA neurons in the substantia nigra of PQ treated rats, their morphology was damaged compared to saline injected animals as there were very few nerve fibers extending from the cell bodies. ASH, while not as effective at maintaining the same amount of DA neurons, better protected the overall morphology of the neurons as there were a lot of fibers that extended off the cell bodies similar to the saline injected animals. When combined, the benefits of Ubisol-Q10 and ASH were seen in the brains of PQ treated rats (neuron numbers maintained by Ubisol-Q10 and cell morphology protected by ASH) (FIG. 1). Furthermore, it was found that the tonic of Ubisol-Q10 and ASH had no ill effects on DA neurons in saline injected rats.

As mentioned before, PD is a multifactorial disease with several biochemical mechanisms including oxidative stress, mitochondrial dysfunction, autophagy impairment, microglia activation, and astroglia inhibition. Ubisol-Q10 was shown to reduce oxidative stress and mitochondria dysfunction in PD cell models. Indeed, we saw that 4-HNE, a lipid peroxidation by-product and oxidative stress marker was almost completely eliminated in the SN of PQ treated rats fed Ubisol-Q10 (FIG. 2). Similarly, ASH also significantly reduced levels of 4-HNE as seen in FIG. 2, confirming the previously reported antioxidant effects of ashwagandha extracts. Furthermore, Ubisol-Q10 fed animals showed elevated levels of beclin-1, a major autophagy regulator, indicating increased levels of autophagy (FIG. 3). These are very exciting results, as upregulation of beclin-1 by Ubisol-Q10 was only previously reported in AD fibroblasts and transgenic AD mice. Thus, the present results further confirm the autophagy activating mechanism of Ubisol-Q10 via upregulation of beclin-1. Activation of autophagy is especially important in PD as defective mitochondria are known to accumulate which can result in further production of ROS leading to apoptosis.

Elevated levels of pro-inflammatory microglia, and reduced activation of pro-survival astrocytes have been implicated in PD. Previously, ashwagandha extracts have been shown to act as an anti-inflammatory in PD. In this study, the status of microglia and astroglia in response to Ubisol-Q10 and ASH treatment were investigated. Confirming previous studies, significant microglia activation was observed in PQ treated rats fed plain water or PTS compared to saline injected animals (FIG. 3). Interestingly, treatment with either Ubisol-Q10, ASH, or the tonic resulted in significant inhibition of active microglia. Furthermore, microglia activation levels had an inverse relationship with levels of 4-HNE. It's possible that microglia could be involved, along with PQ, in inducing oxidative stress. Along with microglia, the status of astroglia was also investigated. Opposite to microglia, reduced levels of active astroglia were observed in PQ treated rats fed plain water or PTS compared to rats injected with saline (FIG. 4). Surprisingly, there was an increase in astroglia activation in both Ubisol-Q10 and especially in ASH (FIG. 4). When combined together, Ubisol-Q10 and ASH were even more potent at inducing astrogliosis compared to the reagents alone. Interestingly, it was even observed that saline injected animals given the tonic had elevated levels of astroglia compared to the saline injected animals fed plain water. Astroglia are also known to secrete several pro-survival neurotrophic factors including GDNF and BDNF. While, both Ubisol-Q10 and ASH resulted in increased astrogliosis, only groups where ASH was given showed increased levels of GDNF and pro-BDNF (precursor to BDNF which eventually gets cleaved to form BDNF) (FIGS. 4 and 5). Without being bound to theory, this could indicate that ASH not only acts as an anti-inflammatory by inhibiting pro-inflammatory microglia and activating pro-survival astroglia but is also stimulating some sort of other pro-survival response.

Previously, it has been reported that CARP1 is thought to be mainly involved in mediating apoptosis. Here, it was observed that CARP1 was down regulated in PQ treated rats given plain water or PTS compared to all other groups (FIG. 2A, 2C), suggesting that CARP1 is acting as a pro-survival regulator in our model of PD. While surprising, this observation is not totally unexpected. CARP1 has been shown to be involved in NR3A synaptic signalling, along with regulating β-catenin in colon cancer metastasis, co-activating GR signaling during adipogenesis, or neurogenin3-mediated pancreatic endocrine differentiation. CARP-1 also interacts with Necdin to regulate myoblast survival. Furthermore, CARP1 interaction with NEMO is also involved in regulation of pro-inflammatory NF-κB survival signalling. CARP1-NEMO signalling is proposed to be involved in regulation of DNA-damage induced survival signalling. CARP-1 is also shown to be a co-activator of the cell cycle regulatory APC/C E3 ligase. APC/C E3 ligase is critical regulator of G2M transition where APC/CCDC20 E3 ligase regulates cyclin B degradation to manage G2 exit. Without being bound to theory, it is possible that elevated levels of CARP-1 in the present study help sustain optimal APC/C CDC20 activity for enhanced G2M exit and cell cycle progression and survival signaling. While a transitory, DNA damage-induced elevated CARP-1 triggers a precipitous decline in cyclin B1 and consequent mitotic crisis as many DNA damaging compounds do cause decline in cyclin B1, G2 arrest, and/or mitotic crisis. Similarly, APC/CCDH1 E3 ligase also targets SCF E3 ligase and together they regulate G1 phase of cell cycle. In both the cases, CARP-1 is a co-activator.

Along with biochemical analysis, animal behaviour in response to PQ insult and the effect the combined tonic of Ubisol-Q10 and ASH has on reducing PQ induced motor deficits were also measured. Seen in FIG. 6, the PQ treated rats given plain drinking water (left) had greater tendency to keep their heads down on the rotorod compared to rats injected with saline and fed plain water (right). Animals given the tonic (center) appeared similar to the saline control animals (right), suggesting that the tonic fed rats were indeed protected from the deleterious effects of PQ. Furthermore, these results also correlate with TH+ staining in FIG. 1, where animals fed the tonic also had higher amounts of intact DA neurons compared to the PQ treated rats fed plain water.

In brief, there are several biochemical mechanisms involved in the progression of PD including oxidative stress, mitochondrial dysfunction, autophagy impairment, and neuroinflammation. Ubisol-Q10 has been shown in the past to protect cultured neurons and rats from PQ induced toxicity by targeting oxidative stress and mitochondria toxicity. Along with targeting oxidative stress and mitochondria dysfunction, we report for the first time that Ubisol-Q10 is able to induce autophagy as well via activation of beclin-1. Furthermore, we showed that ethanolic ashwagandha acted as a potent anti-inflammatory. When combined, these reagents at low doses were even more effective at protecting DA neurons in a PQ induced rat model of PD compared to the reagents alone. Also, for the first time, increased presence of apoptosis regulator CARP1 was observed to be involved in protection/survival of DA neurons. With both Ubisol-Q10 and ASH being simple nutraceutical compounds and GRAS approved, they can be also be taken over long periods of time without serious side effects. Thus, Ubisol-Q10 and ashwagandha root extract could prove to be a promising therapy for PD that could halt neurodegeneration and improve quality of life.

### Example 2—Water-Solubilized Ashwagandha Extract

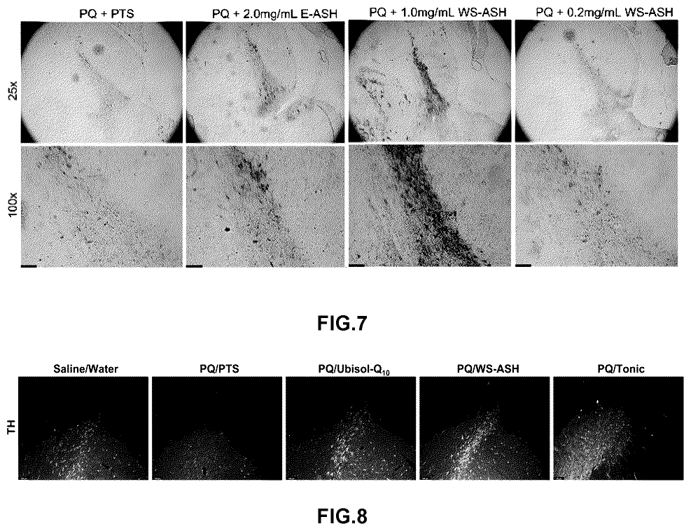

FIG. 7 shows that PTS technology enhances bioavailability and neuroprotection of ethanolic ashwagandha extract. Immunohistochemical staining for tyrosine hydroxylase (TH) indicating dopaminergic neurons in substantia nigra are shown. Rats injected with paraquat (PQ) to stimulate Parkinsonism were given drinking water supplement with either PTS, ethanolic ashwagandha extract (E-ASH) at 120 mg/kg/day, or PTS water-solubilized ethanolic ashwagandha extract (WS-ASH) at either 60 mg/kg/day (water containing 1.0 mg/mL WS-ASH) or 12 mg/kg/day (water containing 0.2 mg/mL WS-ASH). There is a significant enhancement of TH immunoreactivity in rats given the 1.0 mg/mL WS-ASH treatment compared to PTS or the 2.0 mg/mL E-ash. This likely due to enhancement of water-solubility of E-ASH via PTS resulting in increased bioavailability leading to better absorption and neuroprotection. There is a dose dependency with WS-ASH as TH staining decreases as treatment concentration decreases from 1.0 mg/mL WS-ASH to 0.2 mg/mL WS-ASH.).

FIG. 8 shows dopaminergic neurons are better protected by the combination of Ubisol-Q10 combined with WS-ASH compared to agents alone. Immunohistochemical staining for tyrosine hydroxylase (TH) indicating dopaminergic neurons in substantia nigra are shown. Rats were injected with saline and given plain drinking water or paraquat (PQ) to stimulate Parkinsonism and given drinking water supplement with either PTS, PTS water-solubilized coenzyme-Q10 (Ubisol-Q10) at 2 mg/kg/day (water containing 50 μg/mL Ubisol-Q10), PTS water-solubilized ethanolic ashwagandha extract (WS-ASH) at 60 mg/kg/day (water containing 1.0 mg/mL WS-ASH), or the combination of Ubisol-Q10 and WS-ASH at the same above-mentioned doses. There is significant enhancement of TH immunoreactivity in rats given the combination compared to either Ubisol-Q10 or WS-ASH alone. This is likely due to the different neuroprotective properties of Ubisol-Q10 (antioxidant, autophagy activator, mitochondria stabilizer) and WS-ASH (antioxidant, anti-inflammatory) resulting in better neuroprotection.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

1. A formulation comprising:
(i) Coenzyme-Q10 water-solubilized with at least one solubilizing agent of Formula (I): 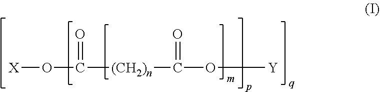
wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
(ii) an alcohol-extracted ashwagandha root extract.

2. A formulation comprising:
(i) an alcohol-extracted ashwagandha root extract water-solubilized with at least one solubilizing agent of Formula (I): 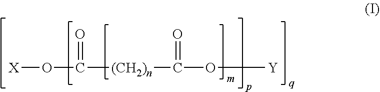
wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2.

3. A formulation comprising:
(i) Coenzyme-Q10 water-solubilized with at least one solubilizing agent of Formula (I): 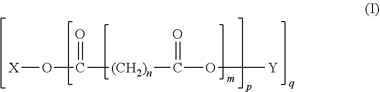
wherein
X is a residue of a hydrophobic moiety selected from sterols, tocopherols and derivatives thereof;
Y is a residue of a hydrophilic moiety selected from polyalcohols, polyethers and derivatives thereof;
m is 0 or 1;
n is an integer of from 0 to 18;
p is 1 or 2; and
q is 1 or 2; and
(ii) an alcohol-extracted ashwagandha root extract water-solubilized with at least one solubilizing agent of Formula (I).

4. The formulation of claim 1, wherein the hydrophobic moiety in Formula (I) is selected from cholesterol, 7-dehydrocholesterol, campesterol, sitosterol, ergosterol, stigmasterol, α-tocopherol, β-tocopherol, γ-tocopherol and δ-tocopherol.

5. The formulation of claim 1, wherein the hydrophobic moiety is α-tocopherol.

6. The formulation of claim 1, wherein the hydrophilic moiety in Formula (I) is a polyether.

7. The formulation of claim 6, wherein the polyether is a polyalkylene glycol.

8. (canceled)

9. The formulation of claim 8, wherein the polyethylene glycol has an average molecular weight of from about 300 to about 5000, or about 400 to about 1000.

10. The formulation of claim 1, wherein the hydrophilic moiety in Formula (I) is a polyalcohol.

11. The formulation of claim 1, wherein when p, m and q are all equal to 1 and the hydrophobic moiety is cholesterol, n is greater than 4 and not equal to 8.

12. The formulation of claim 1, wherein when p, m and q are all equal to 1 and the hydrophobic moiety is α-(+)-tocopherol, n is not equal to 2.

13. The formulation of claim 1, wherein n is an integer of from 2 to 10, or 6 to 10, or 8.

14. The formulation of claim 1, wherein the at least one compound of Formula (I) is polyoxyethanyl-α-tocopheryl sebacate (PTS) monomer, PTS dimer or combinations thereof.

15. The formulation of claim 14, wherein the at least one compound of Formula (I) is PTS monomer.

16. The formulation of claim 14, wherein the at least one compound of Formula (I) is PTS dimer.

17. The formulation of claim 14, wherein the at least one compound of Formula (I) is a combination of PTS monomer and PTS dimer.

18. The formulation of claim 1, wherein the ratio of the solubilizing agent to the Coenzyme-Q10 (w/w) is from about 2:1 to about 12:1 or about 2:1 to 5:1, or about 2:1 or about 3:1.

19. The formulation of claim 1, wherein the ratio of the solubilizing agent to the the alcohol-extracted ashwagandha root extract (w/w) is from about 10:1 to about 1:1, or from about 5:1 to 2:1, or about 3:1 or about 2.8:1.

20. The formulation of claim 1, wherein the formulation contains from about 0.1 mg/mL to about 100 mg/ml of the water solubilized Coenzyme-Q10, or from about 0.1 mg/ml to about 300 mg/ml of the ashwagandha root extract, or from about 0.1 mg/mL to about 20 mg/ml of the water solubilized ashwagandha root extract.

21. The formulation of claim 1, wherein the alcohol-extracted ashwagandha root extract is ethanol-extracted ashwagandha root extract.

22.-28. (canceled)
